(19) United States
(12) Patent Application Publication
(10) Pub. No.: US 2024/0285302 A1
(43) Pub. Date: Aug. 29, 2024

(54) ULTRASOUND TREATMENT TOOL AND METHOD OF MANUFACTURING ULTRASOUND TREATMENT TOOL

(71) Applicant: OLYMPUS MEDICAL SYSTEMS CORP., Tokyo (JP)

(72) Inventors: Yasuhiro MAEDA, Tokyo (JP); Minoru KATSUMATA, Tokyo (JP); Yukihiko SAWADA, Yoshikawa-shi (JP); Hideto YOSHIMINE, Tokyo (JP); Hiroshi ASHIBA, Tokorozawa-shi (JP); Satoshi SHIMABUKURO, Saitama-shi (JP); Shunsuke MATSUI, Tokyo (JP)

(21) Appl. No.: 18/656,132

(22) Filed: May 6, 2024

Related U.S. Application Data
(63) Continuation of application No. PCT/JP2021/041430, filed on Nov. 10, 2021.

(51) Int. Cl. A61B 17/32

(52) U.S. Cl. CPC A61B 17/320092; A61B 2017/320094; A61B 2017/320095

(57) ABSTRACT

An ultrasound treatment tool includes: an ultrasound transducer configured to generate ultrasound vibration; a vibration transmission portion configured to transmit the ultrasound vibration; and a fastener that includes a first fastening portion screwed with the ultrasound transducer and a second fastening portion screwed with the vibration transmission portion, the fastener being configure to connect the ultrasound transducer and the vibration transmission portion. The fastener is configured with a material that has a higher strength than a proximal end portion of the vibration transmission portion, the proximal end portion being screwed with the second fastening portion.

### CROSS-REFERENCE TO RELATED APPLICATION

This application is a continuation of International Application No. PCT/JP2021/041430, filed on Nov. 10, 2021, the entire contents of which are incorporated herein by reference.

### BACKGROUND

### 1. Technical Field

The present disclosure relates to an ultrasound treatment tool and a method of manufacturing an ultrasound treatment tool.

### 2. Related Art

An ultrasound treatment tool that treats a region to be treated (hereinafter, described as a target region) in a living tissue by ultrasound vibration includes an ultrasound transducer that generates the ultrasound vibration and a vibration transmission portion that transmits the ultrasound vibration. The ultrasound transducer and the vibration transmission portion are connected to each other by screwing a male screw portion that is arranged on a proximal end of the vibration transmission portion with a female screw portion that is arranged on a distal end of the ultrasound transducer. Further, conventionally, a technology for manufacturing the vibration transmission portion by shrink fitting is known (for example, International Publication No. WO 2019/116510).

Specifically, the vibration transmission portion described in International Publication No. WO 2019/116510 includes a first rod that is formed in a cylindrical shape, that has a fitting hole on a distal end thereof, and that includes a male screw portion as described above on a proximal end thereof, and a second rod that has a cylindrical shape with a small outer diameter dimension than the first rod. Further, in the technology described in International Publication No. WO 2019/116510, the vibration transmission portion is manufactured by shrink fitting such that a proximal end portion of the second rod is fixed to the fitting hole while the proximal end portion is being inserted in the fitting hole.

Meanwhile, in the vibration transmission portion described in International Publication No. WO 2019/116510, an aluminum alloy or the like that is a material that can easily be heated and expanded at the time of shrink fitting is adopted as the second rod. Further, the aluminum alloy has a relatively low strength.

Therefore, in some cases, a male screw portion included in the second rod with a relatively low strength may be fractured and the fractured male screw portion may remain in a female screw portion of the ultrasound transducer. In this case, when the ultrasound transducer is to be reused, there is a need to perform operation of removing the remaining portion from the female screw portion in the ultrasound transducer, so that it is difficult to improve convenience.

### SUMMARY

In some embodiments, an ultrasound treatment tool includes: an ultrasound transducer configured to generate ultrasound vibration; a vibration transmission portion configured to transmit the ultrasound vibration; and a fastener that includes a first fastening portion screwed with the ultrasound transducer and a second fastening portion screwed with the vibration transmission portion, the fastener being configure to connect the ultrasound transducer and the vibration transmission portion. The fastener is configured with a material that has a higher strength than a proximal end portion of the vibration transmission portion, the proximal end portion being screwed with the second fastening portion.

In some embodiments, provided is a method of manufacturing an ultrasound treatment tool that includes an ultrasound transducer configured to generate ultrasound vibration; and a vibration transmission portion configured to transmit the ultrasound vibration. The method includes: screwing a fastener that is configured with a material that has a higher strength than a proximal end portion of the vibration transmission portion, with the proximal end portion of the vibration transmission portion; and screwing the fastener with the ultrasound transducer. The fastener is screwed with each of the vibration transmission portion and the ultrasound transducer to set a torque at a time of detachment of the fastener from the ultrasound transducer to be smaller than a torque at a time of detachment of the second fastening portion from the vibration transmission portion.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment system according to a first embodiment;

FIG. 2 is a diagram for explaining a configuration of a vibration transmission portion;

FIG. 3 is a diagram for explaining a configuration of a fastener;

FIG. 4 is a diagram for explaining a fastening torque of the fastener with respect to the vibration transmission portion;

FIG. 5 is a diagram for explaining a fastening torque of an ultrasound transducer with respect to the fastener;

FIG. 6 is a diagram for explaining a torque with which the fastener is removed at the side of the ultrasound transducer when the ultrasound transducer is removed from the vibration transmission portion;

FIG. 7 is a diagram for explaining a torque with which the fastener is removed at the side of the vibration transmission portion when the ultrasound transducer is removed from the vibration transmission portion;

FIG. 8 is a diagram for explaining effects of the first embodiment;

FIG. 9 is a diagram for explaining a configuration of a fastener according to a second embodiment;

FIG. 10 is a diagram for explaining a fastened state of the fastener with respect to the vibration transmission portion and the ultrasound transducer;

FIG. 11 is a diagram for explaining a fastened state of the fastener with respect to the vibration transmission portion and the ultrasound transducer; and

FIG. 12 is a diagram for explaining a modification of the first embodiment and the second embodiment.

### DETAILED DESCRIPTION

Modes (hereinafter, embodiments) for carrying out the disclosure will be described below with reference to the drawings. Meanwhile, the disclosure is not limited by the embodiments described below. In addition, in description of the drawings, the same components are denoted by the same reference symbols.

### First Embodiment

### Overall Configuration of Treatment System

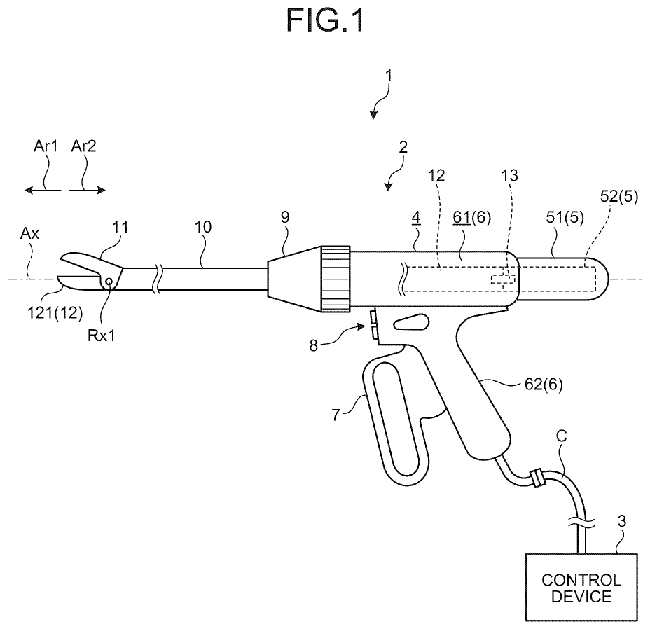

FIG. 1 is a diagram illustrating a treatment system 1 according to one embodiment.

The treatment system 1 applies treatment energy to a region to be treated (hereinafter, described as a target region) in a living tissue, and treats the target region. Meanwhile, the treatment energy in the present embodiment is ultrasound energy and high-frequency energy. Further, treatment that is performed by the treatment system 1 according to the present embodiment is certain treatment, such as coagulation (sealing) of the target region or incision of the target region. Furthermore, it may be possible to adopt a configuration that performs coagulation and incision at the same time. The treatment system 1 includes, as illustrated in FIG. 1, an ultrasound treatment tool 2 and a control device 3.

### Configuration of Ultrasound Treatment Tool

In the following description, in explanation of a configuration of the ultrasound treatment tool 2, one side along a central axis Ax of a shaft 10 is described as a distal end side Ar1, and the other side is described as a proximal end side Ar2.

The ultrasound treatment tool 2 is an ultrasound treatment tool that applies ultrasound energy and high-frequency energy to a target region and treats the target region. The ultrasound treatment tool 2 includes, as illustrated in FIG. 1, a hand piece 4 and an ultrasound transducer unit 5.

The hand piece 4 includes, as illustrated in FIG. 1, a housing 6, a movable handle 7, switches 8, a rotary knob 9, the shaft 10, a jaw 11, a vibration transmission portion 12, and a fastener 13.

The housing 6 supports the entire ultrasound treatment tool 2. The housing 6 includes, as illustrated in FIG. 1, an approximately cylindrical case body 61 that is coaxial with the central axis Ax, and a fixed handle 62 that extends downward from the case body 61 in FIG. 1 and that is gripped by an operating person, such as an operator.

The movable handle 7 receives each of closing operation and opening operation performed by an operating person, such as an operator. Further, the movable handle 7 moves in a direction approaching the fixed handle 62 in accordance with the closing operation. In contrast, the movable handle 7 moves in a direction separating from the fixed handle 62 in accordance with the opening operation.

Each of the switches 8 is exposed to outside from a side face of the fixed handle 62 on the distal end side Ar1 as illustrated in FIG. 1. Further, the switches 8 receive treatment operation performed by an operating person, such as an operator. The treatment operation is operation of applying treatment energy to a target region.

The rotary knob 9 has an approximately cylindrical shape that is coaxial with the central axis Ax, and is arranged on the case body 61 on the distal end side Ar1 as illustrated in FIG. 1. Further, the rotary knob 9 receives rotation operation performed by an operating person, such as an operator. Due to the rotation operation, the rotary knob 9 rotates about the central axis Ax with respect to the case body 61. Furthermore, with the rotation of the rotary knob 9, the shaft 10, the jaw 11, and the vibration transmission portion 12 also rotate about the central axis Ax.

The shaft 10 is a cylindrical pipe that is configured with a certain material, such as a metal.

In the shaft 10, the jaw 11 is pivotally supported so as to be rotatable about a rotation axis Rx1 that extends in a direction perpendicular to the sheet of FIG. 1 on an end portion on the distal end side Ar1.

Here, although specific illustration is omitted, an open-close mechanism that rotates the jaw 11 about the rotation axis Rx1 in accordance with the opening operation and the closing operation performed by an operating person, such as an operator, on the movable handle 7 is arranged inside the housing 6 and the shaft 10.

Further, the jaw 11 is caused to open and close with respect to an end portion 121 (hereinafter, described as the treatment unit 121 (FIG. 1)) of the vibration transmission portion 12 on the distal end side Ar1 by the open-close mechanism, and grips the target region with the treatment unit 121.

The vibration transmission portion 12 is configured with a conductive material, and has an elongated shape that extends in a linear manner along the central axis Ax. Further, as illustrated in FIG. 1, the vibration transmission portion 12 is inserted into the shaft 10 such that the treatment unit 121 protrudes to the outside. In this case, an end portion of the vibration transmission portion 12 on the proximal end side Ar2 is mechanically connected to an ultrasound transducer 52 that constitutes the ultrasound transducer unit 5 by the fastener 13.

Further, the vibration transmission portion 12 transmits ultrasound vibration that is generated by the ultrasound transducer unit 5 from the end portion on the proximal end side Ar2 to the treatment unit 121. In the first embodiment, the ultrasound vibration is longitudinal vibration that vibrates in a direction extending along the central axis Ax.

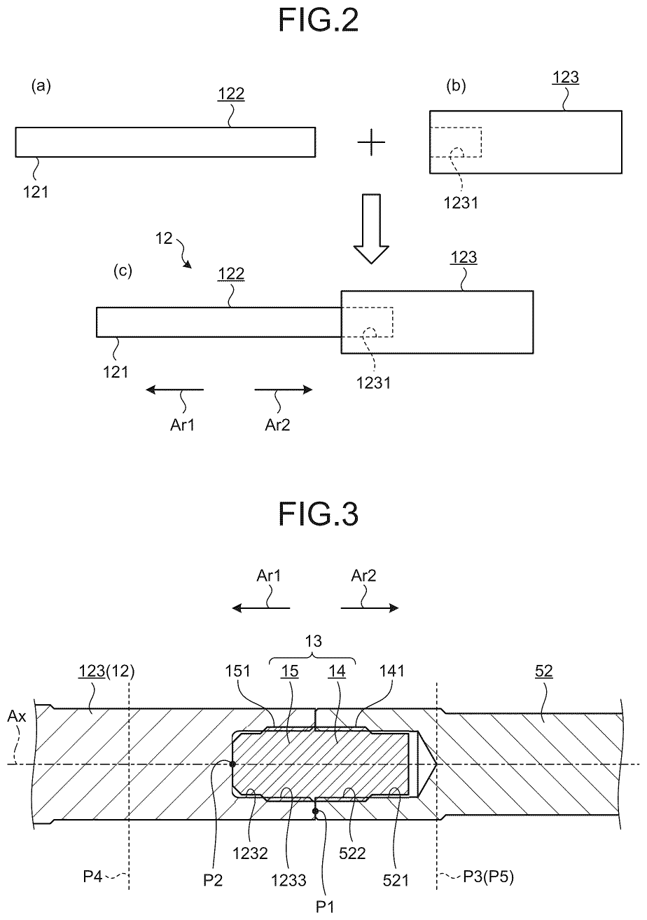

FIG. 2 is a diagram for explaining a configuration of the vibration transmission portion 12.

The vibration transmission portion 12 as described above includes, as illustrated in FIG. 2, two members such as a small diameter portion 122 and a large diameter portion 123.

The small diameter portion 122 is a portion in which an end portion on the distal end side Ar1 serves as the treatment unit 121, is configured with, for example, a titanium alloy, and has a cylindrical shape that linearly extends along the central axis Ax.

The large diameter portion 123 corresponds to a proximal end portion. The large diameter portion 123 is configured with, for example, an aluminum alloy and has a cylindrical shape that linearly extends along the central axis Ax. Here, a concave portion 1231 (FIG. 2) that is recessed toward the proximal end side Ar2 is arranged on an end face of the large diameter portion 123 on the distal end side Ar1. Further, an end portion of the small diameter portion 122 on the proximal end side Ar2 is fixed in a state of being inserted in the concave portion 1231. Examples of a fixing method include a shrink fitting fixing method, a press fitting fixing method (for example, see International Publication No. WO 2019/116510), a screw fitting method, and an adhesive fixing method.

The fastener 13 is a part that connects the vibration transmission portion 12 and the ultrasound transducer 52.

Meanwhile, a specific configuration of the fastener 13 will be described in “configuration of fastener” to be described later.

The ultrasound transducer unit 5 includes, as illustrated in FIG. 1, a transducer (TD) case 51 and the ultrasound transducer 52.

The TD case 51 supports the ultrasound transducer 52 and is detachably connected to the case body 61.

The ultrasound transducer 52 generates ultrasound vibration under the control of the control device 3. In the first embodiment, the ultrasound transducer 52 is configured with a bolt-clamped Langevin-type transducer (BLT).

### Configuration of Control Device

The control device 3 comprehensively controls operations of the ultrasound treatment tool 2 via an electrical cable C (FIG. 1).

Specifically, the control device 3 detects treatment operation that an operating person, such as an operator, has performed on the switches 8 via the electrical cable C. Further, when detecting the treatment operation, the control device 3 applies treatment energy to a target region that is gripped between the jaw 11 and the treatment unit 121 via the electrical cable C. In other words, the control device 3 treats the target region.

For example, when applying ultrasound energy to the target region, the control device 3 supplies driving power to the ultrasound transducer 52 via the electrical cable C. Accordingly, the ultrasound transducer 52 generates longitudinal vibration (ultrasound vibration) that vibrates in a direction extending along the central axis Ax. Further, the treatment unit 121 oscillates at a desired amplitude due to the longitudinal vibration. Furthermore, ultrasound vibration is applied from the treatment unit 121 to the target region that is gripped between the the jaw 11 and the treatment unit 121. In other words, ultrasound energy is applied from the treatment unit 121 to the target region.

Moreover, for example, when applying high-frequency energy to the target region, the control device 3 supplies high-frequency power between the jaw 11 and the vibration transmission portion 12 via the electrical cable C. Accordingly, a high-frequency electric current flows through the target region that is gripped between the jaw 11 and the treatment unit 121. In other words, high-frequency energy is applied to the target region. In this case, at least a part of the jaw 11 is a conductor. Furthermore, the shaft 10 may be configured as a conductor to form a current line.

### Configuration of Fastener

A configuration of the fastener 13 will be described below.

FIG. 3 is a diagram for explaining the configuration of the fastener 13. Specifically, FIG. 3 is a cross-sectional view illustrating a state in which the vibration transmission portion 12 and the ultrasound transducer 52 are connected to each other by the fastener 13.

Meanwhile, in FIG. 3, only a front mass that is arranged on the distal end side Ar1 is illustrated as the ultrasound transducer 52. The front mass is configured with, for example, a titanium alloy.

Here, as illustrated in FIG. 3, a concave portion 521 that is recessed toward the proximal end side Ar2 is arranged on an end face of the ultrasound transducer 52 (front mass) on the distal end side Ar1. The concave portion 521 is a circle for which a cross-section cut along a plane perpendicular to the central axis Ax is coaxial with the central axis Ax, and has approximately the same cross-sectional shape over an entire length along the central axis Ax. Further, a screw groove 522 (FIG. 3) is arranged on an inner peripheral surface of the concave portion 521. Specifically, the concave portion 521 corresponds to a first female screw portion. In the following, for convenience of explanation, the concave portion 521 will be described as the first female screw portion 521.

Furthermore, as illustrated in FIG. 3, a concave portion 1232 that is recessed toward the distal end side Ar1 is arranged on an end face of the large diameter portion 123 on the proximal end side Ar2. The concave portion 1232 is a circle for which a cross-section cut along a plane perpendicular to the central axis Ax is coaxial with the central axis Ax, and has approximately the same cross-sectional shape over an entire length along the central axis Ax. Meanwhile, an inner diameter dimension of the concave portion 1232 is approximately the same as an inner diameter dimension of the first female screw portion 521. Moreover, a screw groove 1233 (FIG. 3) is arranged on an inner peripheral surface of the concave portion 1232. Specifically, the concave portion 1232 corresponds to a second female screw portion. In the following, for convenience of explanation, the concave portion 1232 will be described as the second female screw portion 1232.

The fastener 13 may be configured with any material as long as the material of the fastener 13 has a higher strength than a material that constitutes the large diameter portion 123, and is configured with, for example, a titanium alloy or stainless. As illustrated in FIG. 3, the fastener 13 has a cylindrical shape that is coaxial with the central axis Ax and that extends along the central axis Ax.

A portion of the fastener 13 on the proximal end side Ar2 functions as a first fastening portion 14 (FIG. 3) that is a male screw portion that is screwed with the first female screw portion 521 of the ultrasound transducer 52. Specifically, a screw groove 141 (FIG. 3) is arranged on an outer peripheral surface of the first fastening portion 14. In the first embodiment, a screwed structure between the first female screw portion 521 and the first fastening portion 14 is configured with a screwed structure of a right-handed screw.

Further, a portion of the fastener 13 on the distal end side Ar1 functions as a second fastening portion 15 (FIG. 3) that is a male screw portion that is screwed with the second female screw portion 1232 of the vibration transmission portion 12. Specifically, a screw groove 151 (FIG. 3) is arranged on an outer peripheral surface of the second fastening portion 15. In the first embodiment, a screwed structure between the second female screw portion 1232 and the second fastening portion 15 is configured with a screwed structure of a right-handed screw.

As described above, the fastener 13 has the same structure as what is called a set screw.

### Method of Manufacturing Ultrasound Treatment Tool

A method of manufacturing (method of assembling) the ultrasound treatment tool 2 as described above will be described below.

Meanwhile, in the following, processes for connecting the vibration transmission portion 12 and the ultrasound transducer 52 by the fastener 13 will be mainly described.

First, an operator fastens the fastener 13 to the vibration transmission portion 12 (first fastening process).

Specifically, the operator screws the second fastening portion 15 with the second female screw portion 1232. Further, the operator rotates the fastener 13 about the central axis Ax with respect to the vibration transmission portion 12 by using a torque wrench, and fastens the fastener 13 to the vibration transmission portion 12 with a predetermined torque (corresponding to a fastening torque T to be described later). In this state, an end face of the second fastening portion 15 on the distal end side Ar1 comes into contact with a bottom surface of the second female screw portion 1232 as illustrated in FIG. 3.

Subsequently, the operator incorporates the vibration transmission portion 12 to which the fastener 13 is fastened into the housing 6 and assembles the hand piece 4 (hand piece assembly process).

Further, the operator attaches the ultrasound transducer unit 5 to the case body 61 (transducer attachment process).

Furthermore, the operator connects the vibration transmission portion 12 and the ultrasound transducer 52 to each other by using a torque wrench (second fastening process, for example, see Japanese Patent No. 4675437).

Specifically, the operator engages the torque wrench with the rotary knob 9. Further, the operator rotates the rotary knob 9 about the central axis Ax by using the torque wrench. Accordingly, the vibration transmission portion 12 rotates about the central axis Ax together with the rotary knob 9. Furthermore, the first fastening portion 14 in the fastener 13 that is fastened to the vibration transmission portion 12 is screwed with the first female screw portion 521 until an end face of the vibration transmission portion 12 on the proximal end side Ar2 and an end face of the ultrasound transducer 52 on the distal end side Ar1 come into contact with each other. Moreover, the ultrasound transducer 52 is fastened to the fastener 13 with the predetermined torque (corresponding to the fastening torque T′ to be described later). In this state, an end face of the first fastening portion 14 on the proximal end side Ar2 does not come into contact with a bottom surface of the first female screw portion 521 as illustrated in FIG. 3.

### Connection Portion Between Vibration Transmission Portion and Ultrasound Transducer by Fastener

As described above, in a state in which the vibration transmission portion 12 and the ultrasound transducer 52 are connected to each other by the fastener 13, a positional relationship among the connection portion, an anti-node position, and a node position in the ultrasound vibration (longitudinal vibration) is as follows.

A contact position P1 between the ultrasound transducer 52 and the vibration transmission portion 12 and a position P2 of the bottom surface of the second female screw portion 1232 are located between an anti-node position P3 and a node position P4 (FIG. 3) that are adjacent to each other in order of the anti-node position and the node position from the proximal end side Ar2 to the distal end side Ar1 among anti-node positions and node positions in the ultrasound vibration. Further, a position P5 of the bottom surface of the first female screw portion 521 is located at the anti-node position P3 as illustrated in FIG. 3. In other words, a distance between the position P5 and the anti-node position P3 (zero in the first embodiment) is smaller than a distance between the position P2 and the anti-node position P3.

Here, as described above, the large diameter portion 123 is configured with an aluminum alloy. Further, the fastener 13 is configured with a titanium alloy or stainless. Furthermore, the ultrasound transducer 52 (front mass) is configured with a titanium alloy.

Therefore, the connection portion between the vibration transmission portion 12 and the ultrasound transducer 52 by the fastener 13 is set such that acoustic impedance of components is reduced from the proximal end side Ar2 to the distal end side Ar1.

### Torque at Time of Fastening and Disassembly

A torque at the time of fastening the fastener 13 to the vibration transmission portion 12 and the ultrasound transducer 52, and a torque at the time of disassembling the fastener 13 from the vibration transmission portion 12 and the ultrasound transducer 52 will be described below.

In the following, a fastening torque of the fastener 13 with respect to the vibration transmission portion 12 will be described as a fastening torque T. Further, a fastening torque of the ultrasound transducer 52 with respect to the fastener 13 will be described as a fastening torque T′. Furthermore, a torque with which the fastener 13 is detached at the side of the ultrasound transducer 52 when the ultrasound transducer 52 is removed from the vibration transmission portion 12 will be described as a torque Tᵣ′. Moreover, a torque with which the fastener 13 is detached at the side of the vibration transmission portion 12 when the ultrasound transducer 52 is removed from the vibration transmission portion 12 will be described as a torque Tᵣ.

### Fastening Torque T

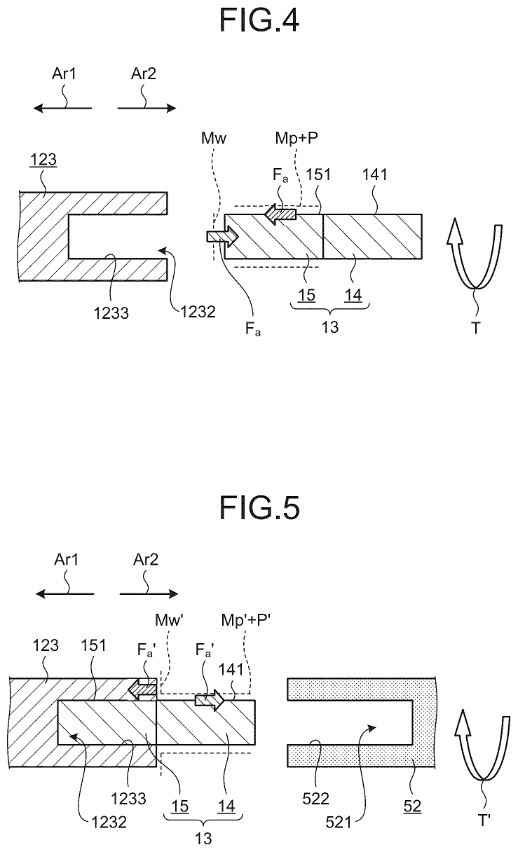

FIG. 4 is a diagram for explaining the fastening torque T of the fastener 13 with respect to the vibration transmission portion 12.

The fastening torque T is calculated by Expression (1) below. In Expression (1), Fₐ represents axial tension. D₂ represents an effective diameter of a screw. μₛ represents a sliding friction coefficient of a screw surface. α represents a half angle of a screw thread. P represents a screw pitch. μ_{w} represents a sliding friction coefficient of a mating surface. D_{w} represents an equivalent friction diameter. The same applies to Expression (8) below.

$$T = {F}_{a} ( \frac{{D}_{2} {μ}_{s}}{2 cos α} + \frac{P}{2 π} + {μ}_{w} \frac{{D}_{w}}{2} ) \qquad ( 1 )$$

In the following, for convenience of explanation, in a parentheses in Expression (1), a first term is represented by Mp, a second term is represented by P, and a third term is represented by Mw. Therefore, Expression (1) is represented by Expression (2) below. Further, in FIG. 4, a portion corresponding to Mp+P and a portion corresponding to Mw are represented by dashed lines, and Fₐ is represented by an arrow.

$$T = {F}_{a} ( Mp + P + Mw ) \qquad ( 2 )$$

Fastening Torque T′

FIG. 5 is a diagram for explaining the fastening torque T′ of the ultrasound transducer 52 with respect to the fastener 13.

The fastening torque T′ is calculated by Expression (3) below. In Expression (3), a prime symbol (′) is added to each of Fₐ, D₂, μₛ, α, P, μ_{w}, and D_{w} to indicate values at the side of the ultrasound transducer 52. The same applies to Expression (6) and Expression (8) below.

$${T}^{′} = {F}_{a}^{′} ( \frac{{D}_{2} {′μ}_{S} ′}{2 cos α′} + \frac{P ′}{2 π} + {μ}_{w}^{′} \frac{{D}_{w} ′}{2} ) \qquad ( 3 )$$

In the following, for convenience of explanation, in a parentheses in Expression (3), a first term is represented by Mp′, a second term is represented by P′, and a third term is represented by Mw′. Therefore, Expression (3) is represented by Expression (4) below. Further, in FIG. 5, a portion corresponding to Mp′+P′ and a portion corresponding to Mw′ are represented by dashed lines, and Fₐ′ is represented by an arrow.

$${T}^{′} = {F}_{a}^{′} ( {Mp}^{′} + {P}^{′} + {Mw}^{′} ) \qquad ( 4 )$$

Furthermore, in the first embodiment, in the method of manufacturing the ultrasound treatment tool 2 as described above, the ultrasound treatment tool 2 is manufactured in a state in which Expression (5) is met.

$$\frac{T}{T ′} \geq 0.33 \qquad ( 5 )$$

Torque Tr′

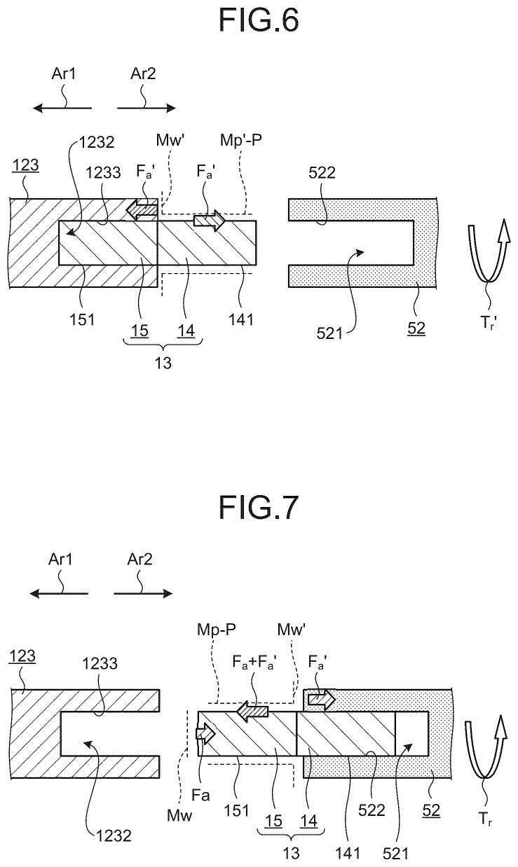

FIG. 6 is a diagram for explaining a torque Tᵣ′ with which the fastener 13 is detached at the side of the ultrasound transducer 52 when the ultrasound transducer 52 is removed from the vibration transmission portion 12.

The torque Tᵣ′ is calculated by Expression (6) below.

$${T}_{r}^{′} = {F}_{a}^{′} ( \frac{{D}_{2} {′μ}_{S} ′}{2 cos α′} - \frac{P ′}{2 π} + {μ}_{w}^{′} \frac{{D}_{w} ′}{2} ) \qquad ( 6 )$$

Further, with use of Mp′, P′, and Mw′ as described above, Expression (6) is represented by Expression (7) below. Furthermore, in FIG. 6, a portion corresponding to Mp′−P′ and a portion corresponding to Mw′ are represented by dashed lines, and Fₐ′ is represented by an arrow.

$${T}_{r}^{′} = {F}_{a}^{′} ( {Mp}^{′} - {P}^{′} + {Mw}^{′} ) \qquad ( 7 )$$

### Torque Tᵣ

FIG. 7 is a diagram for explaining a torque Tᵣ with which the fastener 13 is detached at the side of the vibration transmission portion 12 when the ultrasound transducer 52 is removed from the vibration transmission portion 12.

The torque Tᵣ is calculated by Expression (8) below.

$${T}_{r} = ( {F}_{a} + {F}_{a}^{′} ( \frac{{D}_{2} {μ}_{s}}{2 cos α} - \frac{P}{2 π} ) + {F}_{a} {μ}_{w} \frac{{D}_{w}}{2} + {F}_{a}^{′} {μ}_{w}^{′} \frac{{D}_{w} ′}{2} \qquad ( 8 )$$

Further, with use of Mp, Mp′, P, P′, Mw, and Mw′ as described above, Expression (8) is represented by Expression (9) below. Furthermore, in FIG. 7, a portion corresponding to Mp−P, a portion corresponding to Mw, and a portion corresponding to Mw′ are represented by dashed lines, and Fₐ, Fₐ′, and Fₐ+Fₐ′ are represented by arrows.

$${T}_{r} = ( {F}_{a} + {F}_{a}^{′} ) ( M p - P ) + {F}_{a} Mw + {F}_{a}^{′} {Mw}^{′} \qquad ( 9 )$$

Moreover, in the first embodiment, in the method of manufacturing the ultrasound treatment tool 2 as described above, the ultrasound treatment tool 2 is manufacture in a state in which Expression (10) below is met. Meanwhile, a left side in Expression (10) corresponds to Tᵣ−Tᵣ′. In other words, in the first embodiment, the ultrasound treatment tool 2 is manufactured to set the torque Tᵣ′ at a time of detachment of the fastener 13 from the ultrasound transducer 52 to be smaller than the torque Tᵣ at a time of detachment of the fastener 13 from the vibration transmission portion 12.

$$\frac{( \frac{{D}_{2} {μ}_{s}}{2 cos α} - \frac{P}{2 π} + {μ}_{w} \frac{{D}_{w}}{2} )}{( \frac{{D}_{2} {μ}_{s}}{2 cos α} + \frac{P}{2 π} + {μ}_{w} \frac{{D}_{w}}{2} )} T + \frac{( \frac{{D}_{2} {μ}_{s}}{2 cos α} - \frac{P}{2 π} + \frac{{P}^{′}}{2 π} - \frac{{D}_{2}^{′} {μ}_{s}^{′}}{2 cos {α}^{′}} )}{( \frac{{D}_{2}^{′} {μ}_{s}^{′}}{2 cos {α}^{′}} + \frac{{P}^{′}}{2 π} + {μ}_{w}^{′} \frac{{D}_{w}^{′}}{2} )} {T}^{′} > 0 \qquad ( 10 )$$

Furthermore, in the first embodiment, to meet Expression (10), a state is set such that Expressions (11) to (13) below are met.

$${D}_{2} \geq {D}_{2}^{′} \qquad ( 11 )$$

$${μ}_{s} ≧ {μ}_{s}^{′} \qquad ( 12 )$$

$$P ≦ {P}^{′} \qquad ( 13 )$$

According to the first embodiment as described above, it is possible to achieve effects as described below.

In the ultrasound treatment tool 2 according to the first embodiment, the fastener 13 is configured with a material that has a higher strength than the large diameter portion 123 included in the vibration transmission portion 12. Therefore, the fastener 13 is less likely to be fractured and the fractured fastener 13 is less likely to remain in the first female screw portion 521. In other words, when the ultrasound transducer 52 is to be reused, operation of removing the remaining portion from the first female screw portion 521 is not needed.

Therefore, according to the ultrasound treatment tool 2 of the first embodiment, it is possible to improve convenience.

In the first embodiment, in the method of manufacturing the ultrasound treatment tool 2, the ultrasound treatment tool 2 is manufactured in the state in which Expression (10) is met. Therefore, when the rotary knob 9 is rotated about the central axis Ax and the ultrasound transducer 52 is removed from the vibration transmission portion 12, the fastener 13 is detached at the side of the ultrasound transducer 52 while keeping a state of being fastened to the vibration transmission portion 12. Therefore, when the ultrasound transducer 52 is to be reused, operation of removing the fastener 13 from the first female screw portion 521 is not needed, so that it is possible to further improve convenience.

In particular, in the first embodiment, a state is set such that Expressions (11) to (13) are met. Therefore, it is possible to easily realize a state in which Expression (10) is met.

Furthermore, in the state in which the fastener 13 is fastened to the vibration transmission portion 12, the end face of the second fastening portion 15 on the distal end side Ar1 comes into contact with the bottom surface of the second female screw portion 1232. In contrast, in the state in which the fastener 13 is fastened to the ultrasound transducer 52, the end face of the first fastening portion 14 on the proximal end side Ar2 does not come into contact with the bottom surface of the first female screw portion 521. Therefore, a strong axial tension occurs between the second female screw portion 1232 and the second fastening portion 15, so that it is possible to increase strength of fastening of the fastener 13 to the vibration transmission portion 12 as compared to fastening of the fastener 13 to the ultrasound transducer 52. Therefore, when the ultrasound transducer 52 is removed from the vibration transmission portion 12, it is possible to effectively prevent the fastener 13 from remaining in the ultrasound transducer 52.

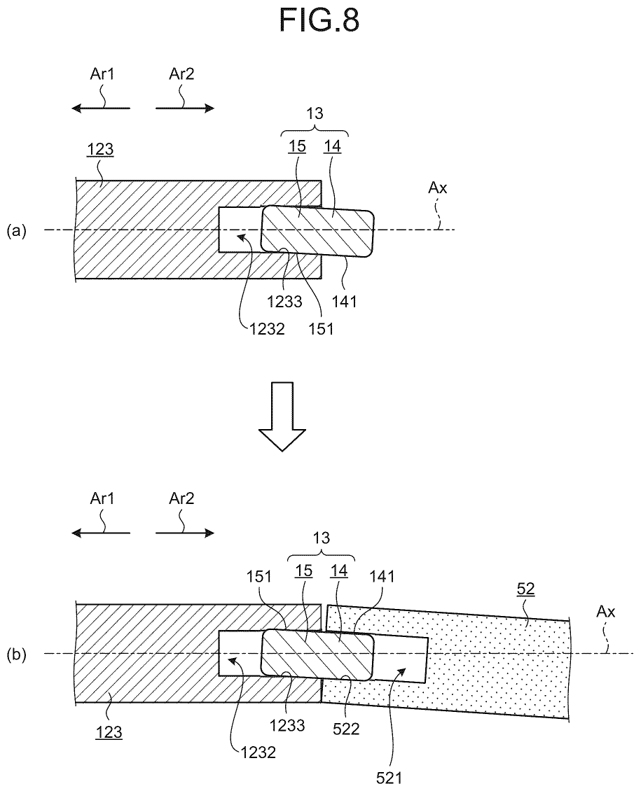

FIG. 8 is a diagram for explaining effects of the first embodiment. Specifically, FIG. 8 is a diagram corresponding to FIG. 3.

Meanwhile, in the state in which the fastener 13 is fastened to the vibration transmission portion 12, if the end face of the second fastening portion 15 on the distal end side Ar1 does not come into contact with the bottom surface of the second female screw portion 1232, a problem as illustrated in FIG. 8 may occur.

Specifically, in the situation as described above, as illustrated in (a) of FIG. 8, the fastener 13 may be fastened to the vibration transmission portion 12 in a posture of being inclined with respect to the central axis Ax. Further, in the state in which the fastener 13 is fastened to the ultrasound transducer 52, as illustrated in (b) of FIG. 8, the ultrasound transducer 52 is similarly inclined with respect to the central axis Ax. In this state, a gap is generated in a part of a contact portion between the vibration transmission portion 12 and the ultrasound transducer 52 and abnormality occurs in transmission of ultrasound vibration, which is a problem.

In contrast, in the first embodiment, in the state in which the fastener 13 is fastened to the vibration transmission portion 12, the end face of the second fastening portion 15 on the distal end side Ar1 comes into contact with the bottom surface of the second female screw portion 1232. The bottom surface of the second female screw portion 1232 is arranged so as to be perpendicular to the central axis Ax, so that the end face of the second fastening portion 15 on the distal end side Ar1 can be fastened in a posture of being perpendicular to the central axis Ax. Therefore, the fastener 13 is not fastened to the vibration transmission portion 12 in the state of being inclined with respect to the central axis Ax, so that the problem as described above can hardly occur.

Meanwhile, a fatigue failure due to ultrasound vibration is likely to occur in a portion corresponding to the screw groove 1233 in the second female screw portion 1232 because of (A) to (C) as described below. If a fatigue failure occurs in the screw groove 1233, fastening to the second fastening portion 15 becomes unstable and ultrasound vibration is not fully transmitted.
(A) A portion corresponding to the screw groove 1233 is located at a position separated from the anti-node position P3, so that stress applied by the ultrasound vibration is increased.
(B) Stress is likely to be concentrated in a screw shape.
(C) The second female screw portion 1232 is configured with an aluminum alloy having a low strength.

In contrast, in the first embodiment, in the state in which the fastener 13 is fastened to the vibration transmission portion 12, the end face of the second fastening portion 15 on the distal end side Ar1 comes into contact with the bottom surface of the second female screw portion 1232. Therefore, it is possible to generate a strong axial tension in the portion corresponding to the screw groove 1233, so that it is possible to reduce a stress amplitude and realize a structure in which a fatigue failure is less likely to occur in the portion.

In particular, in the first embodiment, in the method of manufacturing the ultrasound treatment tool 2, the ultrasound treatment tool 2 is manufacture in a state in which Expression (5) is met. Therefore, it is possible to generate a fully strong axial tension in the portion corresponding to the screw groove 1233.

Furthermore, another effect that is achieved by manufacturing the ultrasound treatment tool 2 in the state in which Expression (5) is met in the method of manufacturing the ultrasound treatment tool 2 was confirmed by an experiment as described below.

Specifically, in two cases in one of which T/T′ is set to 0.27, and in the other one of which T/T′ is set to 0.33, the ultrasound transducer 52 was removed from the vibration transmission portion 12 and it was confirmed whether the fastener 13 was detached at the side of the vibration transmission portion 12 or the fastener 13 was detached at the side of the ultrasound transducer 52. Meanwhile, in the two cases as described above, the fastening torque T′ was set to the same. A result is as indicated by Table 1 below. Meanwhile, in Table 1, “OK” indicates a case in which the fastener 13 was detached at the side of the vibration transmission portion 12, and “NG” indicates a case in which the fastener 13 was detached at the side of the ultrasound transducer 52.

TABLE 1

| T/T′ | Result |
|---|---|
| 0.27 | NG |
| 0.33 | OK |

As indicated in Table 1, by manufacturing the ultrasound treatment tool 2 in the state in which Expression (5) is met, it is possible to effectively prevent the fastener 13 from remaining in the ultrasound transducer 52 when the ultrasound transducer 52 is removed from the vibration transmission portion 12.

Meanwhile, for example, it is assumed that the vibration transmission portion 121 includes the small diameter portion 122 and the large diameter portion 123 that are integrally formed form a single material. In the vibration transmission portion 12 as described above, by arranging a tapered portion in which a cross sectional area is reduced from the proximal end side Ar2 to the distal end side Ar1 in the large diameter portion 123, it is possible to increase an amplitude of the ultrasound vibration in the tapered portion.

In contrast, in the vibration transmission portion 12 according to the first embodiment, the small diameter portion 122 and the large diameter portion 123 are fixed by a certain fixing method, such as shrink fitting or press fitting. Further, it is difficult to arrange the tapered portion as described above in the large diameter portion 123 when the fixing states of the small diameter portion 122 and the large diameter portion 123 are taken into account. In other words, it is needed to arrange the tapered portion as described above in the small diameter portion 122. However, a cross-sectional area of the small diameter portion 122 is originally small. Therefore, even if the tapered portion as described above is arranged in the small diameter portion 122, a total magnification ratio for increasing the amplitude of the ultrasound vibration may be insufficient. Furthermore, by arranging the tapered portion in the small diameter portion 122, the cross-sectional area of the small diameter portion 122 is further reduced, so that rigidity of the small diameter portion 122 may be reduced and abnormal vibration, other than longitudinal vibration, may occur at the time of occurrence of the ultrasound vibration.

To cope with this, in the first embodiment, by using the fact that an amplitude increases at a boundary position between different kinds of materials due to differences in physical properties (in particular, acoustic impedance), an amplitude of ultrasound vibration is increased in the vibration transmission portion 12.

Specifically, the contact position P1 between the ultrasound transducer 52 and the vibration transmission portion 12 and the position P2 at which the end face of the second fastening portion 15 on the distal end side Ar1 comes into contact with the bottom surface of the second female screw portion 1232 correspond to boundary positions. Further, each of the positions P1 and P2 is located between the anti-node position P3 and the node position P4 that are adjacent to each other in order of the anti-node position and the node position from the proximal end side Ar2 to the distal end side Ar1 among anti-node positions and node positions in the ultrasound vibration. In other words, the positions P1 and P2 are located at positions separated from the anti-node position P3 to the node position P4. Further, the connection portion between the vibration transmission portion 12 and the ultrasound transducer 52 by the fastener 13 is set such that acoustic impedance of components is reduced from the proximal end side Ar2 to the distal end side Ar1.

Therefore, at each of the positions P1 and P2, it is possible to increase the amplitude of the ultrasound vibration. In other words, even when the vibration transmission portion 12 is configured such that the small diameter portion 122 and the large diameter portion 123 are fitted by a certain fitting method, such as shrink fitting or press fitting, it is possible to fully ensure a total magnification ratio for increasing the amplitude of the ultrasound vibration. Furthermore, a tapered portion need not be arranged in the small diameter portion 122, so that it is possible to prevent reduction in rigidity of the small diameter portion 122.

### Second Embodiment

A second embodiment will be described below.

In the explanation below, the same components as those of the first embodiment as described above are denoted by the same reference symbols, and detailed explanation thereof will be omitted or simplified.

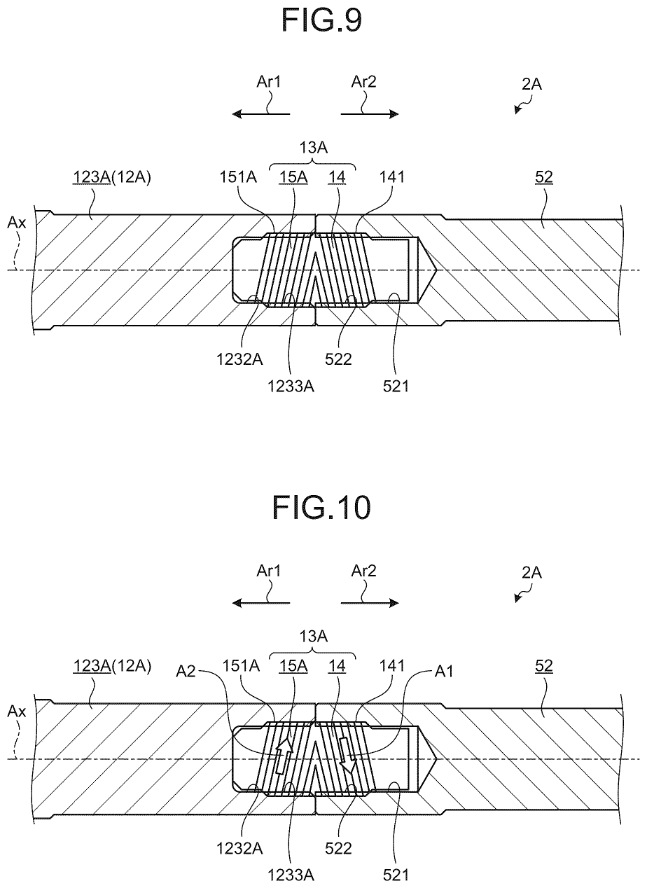

FIG. 9 is a diagram for explaining the second embodiment. Specifically, FIG. 9 is a cross-sectional view corresponding to FIG. 3.

As illustrated in FIG. 9, the second embodiment is different from the first embodiment as described above in that a screwed structure of the second female screw portion 1232 and the second fastening portion 15 is configured with a screwed structure of a left-handed screw. In the following, for convenience of explanation, an ultrasound treatment tool, a vibration transmission portion, a large diameter portion, a second female screw portion (including a screw groove), a fastener, and a second fastening portion (including a screw groove) according to the present embodiment will be described as an ultrasound treatment tool 2A, a vibration transmission portion 12A, a large diameter portion 123A, a second female screw portion 1232A (including a screw groove 1233A), a fastener 13A, and a second fastening portion 15A (including a screw groove 151A), respectively.

A fastened state of the fastener 13A with respect to the vibration transmission portion 12A and the ultrasound transducer 52 will be described below.

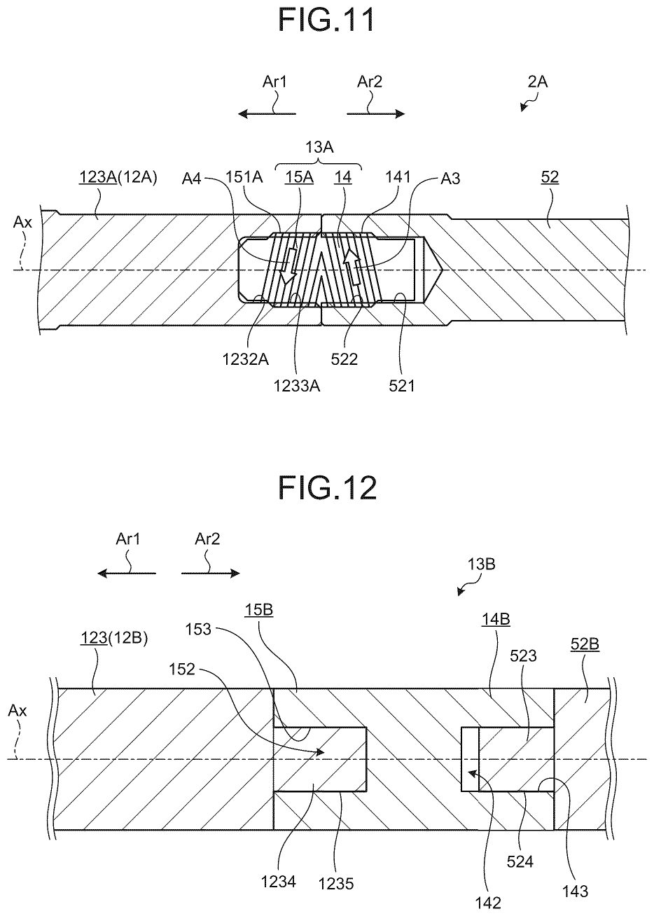

FIG. 10 and FIG. 11 are diagrams for explaining the fastened state of the fastener 13A with respect to the vibration transmission portion 12A and the ultrasound transducer 52. Specifically, FIG. 10 and FIG. 11 are cross-sectional views corresponding to FIG. 3. Meanwhile, in FIG. 10 and FIG. 11, for convenience of explanation, only the fastener 13A is not illustrated by a cross-section.

First, with reference to FIG. 10, a fastened state in a second fastening process that is performed after the first fastening process, the hand piece assembly process, and the transducer attachment process explained in the first embodiment as described above will be described below.

In the second fastening process, the operator rotates the rotary knob 9 in a clockwise direction about the central axis Ax when viewed from the distal end side Ar1. Here, a screwed structure of the first female screw portion 521 and the first fastening portion 14 is configured with a screwed structure of a right-handed screw. Therefore, the first fastening portion 14 in the fastener 13A that is fastened to the vibration transmission portion 12A rotates in a fastening direction as indicated by an arrow A1 in FIG. 10 with respect to the first female screw portion 521. Further, the ultrasound transducer 52 is fastened to the fastener 13A.

Here, the screwed structure between the second female screw portion 1232A and the second fastening portion 15A is configured with a screwed structure of a left-handed screw. Therefore, in the second fastening process, if an excessive torque is added to the fastener 13A, the second fastening portion 15A rotates in a loosening direction as indicated by an arrow A2 in FIG. 10 with respect to the second female screw portion 1232A and is removed from the second female screw portion 1232A. With this configuration, in the second fastening process, even when the rotary knob 9 is rotated about the central axis Ax, the vibration transmission portion 12A spins with respect to the ultrasound transducer 52. In other words, the ultrasound treatment tool 2A is not usable.

A fastened state when the ultrasound transducer 52 is removed from the vibration transmission portion 12A will be described below with reference to FIG. 11.

In this case, the operator rotates the rotary knob 9 in a counterclockwise direction about the central axis Ax when viewed from the distal end side Ar1. Here, the screwed structure between the first female screw portion 521 and the first fastening portion 14 is configured with a screwed structure of a right-handed screw. Therefore, the first fastening portion 14 in the fastener 13A that is fastened to the vibration transmission portion 12A rotates in a loosening direction as indicated by an arrow A3 in FIG. 11 with respect to the first female screw portion 521, and is removed from the second female screw portion 1232A.

Here, the screwed structure between the second female screw portion 1232A and the second fastening portion 15A is configured with a screwed structure of a left-handed screw. Therefore, if the rotary knob 9 is rotated in a counterclockwise direction about the central axis Ax when viewed from the distal end side Ar1 as described above, the second fastening portion 15A rotates in a loosening direction as indicated by an arrow A4 in FIG. 11 with respect to the second female screw portion 1232A, so that a fastened state with respect to the second female screw portion 1232A is increased.

According to the second embodiment as described above, it is possible to achieve effects as described below in addition to achieving the same effects as those of the first embodiment as described above.

In the second embodiment, the screwed structure between the first female screw portion 521 and the first fastening portion 14 is configured with a screwed structure of a right-handed screw. In contrast, the screwed structure between the second female screw portion 1232A and the second fastening portion 15A is configured with a screwed structure of a left-handed screw.

Therefore, when the ultrasound transducer 52 is removed from the vibration transmission portion 12A, the second fastening portion 15A rotates in the loosening direction as indicated by the arrow A4 in FIG. 11 with respect to the second female screw portion 1232A, so that the fastened state with respect to the second female screw portion 1232A is increased. Therefore, it is possible to effectively prevent the fastener 13A from remaining in the ultrasound transducer 52.

Meanwhile, for example, when the first fastening portion 14 is fastened to the ultrasound transducer 52 with a certain torque larger than a specific torque, treatment performance for the target region changes from the desired performance.

In contrast, in the second embodiment, if an excessive torque is applied to the fastener 13A in the second fastening process, the second fastening portion 15A rotates in the loosening direction as indicated by the arrow A2 in FIG. 10 with respect to the second female screw portion 1232A, and is removed from the second female screw portion 1232A. Therefore, the ultrasound treatment tool 2A becomes not usable.

Therefore, when the fastener 13A is fastened to the ultrasound transducer 52 by wrong operation, it is possible to set the ultrasound treatment tool 2A to unusable state.

In the second embodiment as described above, the screwed structure between the first female screw portion 521 and the first fastening portion 14 is configured with a screwed structure of a right-handed screw, and the screwed structure between the second female screw portion 1232A and the second fastening portion 15A is configured with a screwed structure of a left-handed screw, but embodiments are not limited to this example. For example, it may be possible to configure the screwed structure between the first female screw portion 521 and the first fastening portion 14 with a screwed structure of a left-handed screw, and configured the screwed structure between the second female screw portion 1232A and the second fastening portion 15A with a screwed structure of a right-handed screw.

### Other Embodiments

While the embodiments of the disclosure have been described above, the disclosure is not limited by only the first embodiment and the second embodiment as described above.

In the first embodiment and the second embodiment as described above, the ultrasound treatment tool according to the disclosure is configured to apply both of ultrasound energy and high-frequency energy to a target region, but embodiments are not limited to this example. As the ultrasound treatment tool according to the disclosure, it may be possible to adopt a configuration that applies only ultrasound energy to a target region, or it may be possible to adopt a configuration that applies at least one of high-frequency energy and thermal energy to a target region in addition to applying ultrasound energy. Here, “application of thermal energy to a target region” means transmission of heat that is generated by a heater or the like to the target region.

In the first embodiment and the second embodiment as described above, it may be possible to change the fastened structures of the vibration transmission portions 12 and 12A, the ultrasound transducer 52, and the fasteners 13 and 13A. In the following, for convenience of explanation, a vibration transmission portion, an ultrasound transducer, and a fastener according to the present modification will be described as a vibration transmission portion 12B, an ultrasound transducer 52B, and a fastener 13B, respectively.

FIG. 12 is a diagram for explaining a modification of the first embodiment and the second embodiment. Specifically, FIG. 12 is a cross-sectional view of a fastened structure of the vibration transmission portion 12B, the ultrasound transducer 52B, and the fastener 13B when viewed from the side.

In the ultrasound transducer 52B according to the present modification, a structure of an end portion on the distal end side Ar1 is different from the ultrasound transducer 52 that is explained in the first embodiment and the second embodiment as described above.

Specifically, the first female screw portion 521 that is explained in the first embodiment and the second embodiment as described above is not arranged on an end portion of the ultrasound transducer 52B on the distal end side Ar1. Further, on an end face of the ultrasound transducer 52B on the distal end side Ar1, as illustrated in FIG. 12, a cylindrical protruding portion 523 that is coaxial with the central axis Ax and that protrudes toward the distal end side Ar1 along the central axis Ax is arranged. Furthermore, a screw groove 524 (FIG. 12) is arranged on an outer peripheral surface of the protruding portion 523. In other words, the protruding portion 523 functions as a male screw portion. In the following, for convenience of explanation, the protruding portion 523 will be described as a first male screw portion 523.

In the vibration transmission portion 12B according to the present modification, a structure of an end portion on the proximal end side Ar2 is different from the vibration transmission portions 12 and 12A explained in the first embodiment and the second embodiment as described above.

Specifically, the second female screw portions 1232 and 1232A that are explained in the first embodiment and the second embodiment as described above are not arranged in the end portion of the vibration transmission portion 12B on the proximal end side Ar2. Further, a cylindrical protruding portion 1234 that is coaxial with the central axis Ax and that protrudes toward the proximal end side Ar2 along the central axis Ax is arranged on the end face of the vibration transmission portion 12B on the proximal end side Ar2 as illustrated in FIG. 12. Furthermore, a screw groove 1235 (FIG. 12) is arranged on the outer peripheral surface of the protruding portion 1234. In other words, the protruding portion 1234 functions as a male screw portion. In the following, for convenience of explanation, the protruding portion 1234 will be described as a second male screw portion 1234.

The fastener 13B according to the present modification has a cylindrical shape that is coaxial with the central axis Ax and that extends along the central axis Ax.

In the fastener 13B, a portion on the proximal end side Ar2 functions as a first fastening portion 14B (FIG. 12) that is a female screw portion that is screwed with the first male screw portion 523 of the ultrasound transducer 52B.

Specifically, as illustrated in FIG. 12, a concave portion 142 that is recessed toward the distal end side Ar1 is arranged on an end face of the fastener 13B on the proximal end side Ar2. The concave portion 142 is a circle for which a cross-section cut along a plane perpendicular to the central axis Ax is coaxial with the central axis Ax, and has approximately the same cross-sectional shape over an entire length along the central axis Ax. Meanwhile, an inner diameter dimension of the concave portion 142 is approximately the same as an outer diameter dimension of the first male screw portion 523. Further, a screw groove 143 (FIG. 12) is arranged on an inner peripheral surface of the concave portion 142.

Furthermore, in the fastener 13B, a portion on the distal end side Ar1 functions as a second fastening portion 15B that is a female screw portion that is screwed with the second male screw portion 1234 of the vibration transmission portion 12B.

Specifically, as illustrated in FIG. 12, a concave portion 152 that is recessed toward the proximal end side Ar2 on the end face of the fastener 13B on the distal end side Ar1. The concave portion 152 is a circle for which a cross-section cut along a plane perpendicular to the central axis Ax is coaxial with the central axis Ax, and has approximately the same cross-sectional shape over an entire length along the central axis Ax. Meanwhile, an inner diameter dimension of the concave portion 152 is approximately the same as an outer diameter dimension of the second male screw portion 1234. Furthermore, a screw groove 153 (FIG. 12) is arranged on an inner peripheral surface of the concave portion 152.

In the first embodiment and the second embodiment as described above, as the fastened structures of the vibration transmission portions 12 and 12A, the ultrasound transducer 52, and the fasteners 13 and 13A, only one of the fastened structure of the vibration transmission portions 12 and 12A and the fasteners 13 and 13A and the fastening structure of the ultrasound transducer 52 and the fasteners 13 and 13A may be configured as the structure described in the modification as illustrated in FIG. 12.

According to an ultrasound treatment tool and a method of manufacturing the ultrasound treatment tool of the disclosure, it is possible to improve convenience.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound treatment tool comprising:
an ultrasound transducer configured to generate ultrasound vibration;
a vibration transmission portion configured to transmit the ultrasound vibration; and
a fastener that includes a first fastening portion screwed with the ultrasound transducer and a second fastening portion screwed with the vibration transmission portion, the fastener being configure to connect the ultrasound transducer and the vibration transmission portion, wherein
the fastener is configured with a material that has a higher strength than a proximal end portion of the vibration transmission portion, the proximal end portion being screwed with the second fastening portion.

2. The ultrasound treatment tool according to claim 1, wherein a toque at a time of detachment of the first fastening portion from the ultrasound transducer is set to be smaller than a torque at a time of detachment of the second fastening portion from the vibration transmission portion.

3. The ultrasound treatment tool according to claim 2, wherein
the proximal end portion includes a second female screw portion that is recessed toward a distal end of the vibration transmission portion, and
the second fastening portion is a male portion that is screwed with the second female screw portion and is set to be in contact with a bottom surface of the second female screw portion.

4. The ultrasound treatment tool according to claim 3, wherein
a first female screw portion that is recessed toward a proximal end of the ultrasound transducer is arranged in a distal end portion of the ultrasound transducer, the distal end portion being screwed with the first fastening portion, and
the first fastening portion is a male screw portion screwed with the first female screw portion and is set to be in no contact with a bottom surface of the first female screw portion.

5. The ultrasound treatment tool according to claim 4, wherein
a distal end of the ultrasound transducer and a proximal end of the vibration transmission portion are in contact with each other when the ultrasound transducer and the vibration transmission portion are connected to each other by the fastener, and
a contact portion between the ultrasound transducer and the vibration transmission portion and the bottom surface of the female screw portion of the vibration transmission portion are located between an anti-node position and a node position that are adjacent to each other in order of the anti-node position and the node position from a proximal end side to a distal end side of the ultrasound treatment tool among anti-node positions and node positions in the ultrasound vibration.

6. The ultrasound treatment tool according to claim 5, wherein a distance between the bottom surface of the female screw portion of the ultrasound transducer and the anti-node position among the anti-node position and the node position that are adjacent to each other is smaller than a distance between the bottom surface of the female screw portion of the vibration transmission portion and the anti-node position among the anti-node position and the node position that are adjacent to each other.

7. The ultrasound treatment tool according to claim 6, wherein the bottom surface of the female screw portion of the ultrasound transducer is located at the anti-node position among the anti-node position and the node position that are adjacent to each other.

8. The ultrasound treatment tool according to claim 5, wherein a connection portion between the ultrasound transducer and the vibration transmission portion by the fastener is set to reduce an acoustic impedance of components from the proximal end side toward the distal end side.

9. The ultrasound treatment tool according to claim 1, wherein a fastening torque of the fastener with respect to the vibration transmission portion is set to be equal to or larger than 0.33 times of a fastening torque of the fastener with respect to the ultrasound transducer.

10. The ultrasound treatment tool according to claim 1, wherein the proximal end portion is made of an aluminum alloy.

11. The ultrasound treatment tool according to claim 1, wherein one of a first screwed structure between the ultrasound transducer and the first fastening portion and a second screwed structure between the vibration transmission portion and the second fastening portion is a screwed structure of a right-handed screw and another one of the first screwed structure and the second screwed structure is a screwed structure of a left-handed screw.

12. A method of manufacturing an ultrasound treatment tool that includes
an ultrasound transducer configured to generate ultrasound vibration; and
a vibration transmission portion configured to transmit the ultrasound vibration,
the method comprising:
screwing a fastener that is configured with a material that has a higher strength than a proximal end portion of the vibration transmission portion, with the proximal end portion of the vibration transmission portion; and
screwing the fastener with the ultrasound transducer, wherein
the fastener is screwed with each of the vibration transmission portion and the ultrasound transducer to set a torque at a time of detachment of the fastener from the ultrasound transducer to be smaller than a torque at a time of detachment of the second fastening portion from the vibration transmission portion.

13. The method according to claim 12, wherein
the proximal end portion includes a second female screw portion that is recessed toward a distal end of the vibration transmission portion,
the fastener includes a second fastening portion that is a male screw portion screwed with the second female screw portion, and
the screwing the fastener with the vibration transmission portion includes inserting the second fastening portion into the second female screw portion to screw the second fastening portion with the second female screw portion and to bring the second fastening portion into contact with a bottom surface of the second female screw portion.

14. The method according to claim 13, wherein
a first female screw portion that is recessed toward a proximal end of the ultrasound transducer is arranged in a distal end portion of the ultrasound transducer,
the fastener includes a first fastening portion that is a male screw portion screwed with the first female screw portion, and
the screwing the fastener with the ultrasound transducer includes inserting the first fastening portion into the first female screw portion to screw the first fastening portion with the first female screw portion and to prevent the first fastening portion from coming into contact with a bottom surface of the first female screw portion.
